(19) United States
(12) Patent Application Publication
(10) Pub. No.: US 2021/0348219 A1
(43) Pub. Date: Nov. 11, 2021

(54) TARGET NUCLEIC ACID DETECTION METHOD BASED ON PROXIMITY PROTEOLYSIS REACTION

(71) Applicant: AJOU UNIVERSITY INDUSTRY-ACADEMIC COOPERATION FOUNDATION, Suwon-si, Gyeonggi-do (KR)

(72) Inventors: Tae Hyeon YOO, Yongin-si, Gyeonggi-do (KR); Hyeon Ji PARK, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 17/282,650

(22) PCT Filed: Oct. 2, 2019
(86) PCT No.: PCT/KR2019/012891

§ 371 Date: Apr. 2, 2021

(30) Foreign Application Priority Data
Oct. 5, 2018 (KR) 10-2018-0119010

(51) Int. Cl. C12Q 1/6834; G01N 33/53; C12Q 1/6818; G01N 21/64; G01N 21/31

(52) U.S. Cl. CPC C12Q 1/6834; G01N 33/5308; G01N 21/31; G01N 21/6428; C12Q 1/6818

(57) ABSTRACT

A method for detecting a target nucleic acid includes: (a) a step of mixing a sample containing the target nucleic acid with a nucleic acid detection solution containing i) ssDNA-protease conjugate, ii) ssDNA-zymogen conjugate, and iii) a substrate specific for the zymogen; and (b) a step of detecting a signal generated by a proximity proteolysis reaction between the ssDNA-zymogen conjugate and the ssDNA-protease conjugate which are hybridized to the target nucleic acid.

### TECHNICAL FIELD

The present invention relates to a method for detecting a target nucleic acid based on a proximity proteolysis reaction, and more specifically to a method for detecting a target nucleic acid including detecting a signal generated by a proximity proteolysis reaction between a ssDNA-protease conjugate and a ssDNA-zymogen conjugate which are hybridized to the target nucleic acid.

### BACKGROUND ART

Nucleic acid provides a large amount of biological information, and various methods are utilized to use the same. In particular, the concentration of a specific nucleic-acid molecule may be an important indicator of a specific disease condition, and the development of an efficient and simple method for determining the concentration of target DNA or RNA has been intensively researched for several decades (M. Wang, et al., Biotechnology and Bioprocess Engineering 2017, 22, 95-99). Various methods for conversion of hybridization to detectable signals such as absorbance, fluorescent, luminescent and electrochemical signals have been devised using a simple principle of producing a probe specific to a target nucleic acid based on Watson-Crick base pairing (L. Yan, et al., Molecular Biosystems 2014, 10, 970-1003; Y. V. Gerasimova, D. M. Kolpashchikov, Chemical Society Reviews 2014, 43, 6405-6438; X. Su, et al., Applied Spectroscopy 2012, 66, 1249-1262). Most studies have focused on methods based on fluorescent, luminescent and electrochemical signals due to the high sensitivity thereof.

However, most of current fluorescence signal-based nucleic acid detection methods require expensive equipment and analytic samples and highly skilled operators, thus having drawbacks of being performed only at specialized testing institutions, and taking considerable time and incurring considerable expense from collecting samples to obtaining test results. Most current electrochemical signal-based nucleic acid detection methods are signal generation methods using an oxidation/reduction enzyme (Patolsky, et al., Angew. Chem. Int. 2002, 41, 3398), and are disadvantageous in that the total analysis time is increased due to the large number of reaction steps, and signal measurement is possible only during the enzymatic catalytic reaction, and is impossible after completion of the enzymatic catalytic reaction. The nucleic acid detection method based on the absorbance signal has an advantage of being simpler than methods using other signals, for example, a detection instrument therefor is simpler, which is an important factor in developing a field diagnosis method.

Several signal amplification methods have been reported to overcome the low sensitivity limit of the absorbance signal (Y. Guo, et al., Biosensors & Bioelectronics 2017, 94, 651-656), but the addition of a multi-step or time-consuming process inevitably increases complexity.

When nucleic acids are extracted from biological samples such as blood for analysis of nucleic acids (DNA or RNA) in living organisms, the amount of nucleic acids that is extracted is generally too small to be used directly for a variety of analyses, so it is necessary to amplify the extracted nucleic acids in order to accurately analyze the same. Nucleic acid amplification techniques using various methods have been developed to date. In particular, PCR (polymerase chain reaction), which is a representative method of amplifying DNA, is a highly efficient amplification technique that selectively amplifies large amounts of target genes, and thus is widely used in a variety of fields. However, PCR has a disadvantage in that it is expensive and difficult to use for on-site diagnosis, since it requires a PCR device that finely controls the temperature of the reaction solution.

The reaction rate can be improved by placing the reactants close to each other. This principle has been used to detect the target molecule. The best example is the proximity ligation assay: two DNA molecules conjugated to different antibodies are brought into close proximity with each other to allow antigen or protein-protein interactions to occur and can participate in the amplification process of rolling circle DNA synthesis. In addition, this principle is applied to detect various molecules such as proteins (T. E. Schaus, et al., Nature Communications 2017, 8, 696), antibodies (A. Porchetta, et al., Journal of the American Chemical Society 2018, 140, 947-953) and nucleic acids (W. A. Velema, E. T. Kool, Journal of the American Chemical Society 2017, 139, 5405-5411).

Accordingly, as a result of extensive efforts to develop a method for detecting target nucleic acids that is fast, simple, and enables easy detection even at a low nucleic acid concentration, the present inventors found that when the target nucleic acid is detected using a proximity proteolysis reaction of a ssDNA-protease conjugate, in which ssDNA having a sequence complementary to the target nucleic acid is bound to a protease, and a ssDNA-zymogen conjugate, in which ssDNA having a sequence complementary to the target nucleic acid is bound to a zymogen, nucleic acid detection is possible even at a concentration of about 100 pM, is realized through a one-step process of adding two DNA-protein conjugates and a colorimetric substrate to a sample, and is completed within 1 hour. Based on this finding, the present invention has been completed.

The information disclosed in this Background section is provided only for better understanding of the background of the present invention, and therefore it may not include information that forms the prior art that is already obvious to those skilled in the art.

### SUMMARY OF THE INVENTION

It is one object of the present invention to provide a method for detecting target nucleic acids that is fast, simple, and enables easy detection even at a small nucleic acid concentration.

It is another object of the present invention to provide a nucleic acid detection solution used for the method for detecting target nucleic acids.

To achieve the above objects, the present invention provides a method for detecting a target nucleic acid, the method including: (a) mixing a sample containing the target nucleic acid with a nucleic acid detection solution containing: i) a ssDNA-protease conjugate in which ssDNA having a sequence complementary to the target nucleic acid is bound to a protease; ii) a ssDNA-zymogen conjugate in which ssDNA having a sequence complementary to the target nucleic acid is bound to a zymogen; and iii) a substrate specific for the zymogen; and (b) detecting a signal generated by a proximity proteolysis reaction between the ssDNA-zymogen conjugate and the ssDNA-protease conjugate which are hybridized to the target nucleic acid.

The present invention also provides the nucleic acid detection solution used for the method for detecting a target nucleic acid.

### DESCRIPTION OF DRAWINGS

FIG. 1 is a schematic diagram showing a method of detecting a target nucleic acid using a proximity proteolysis reaction according to the present invention.

FIG. 2a is a schematic diagram showing a process of producing a ssDNA-zymogen conjugate, and FIG. 2b is a schematic diagram showing the process of producing a ssDNA-protease conjugate.

FIG. 3a shows a one-step method and analysis results for DNA detection according to the present invention, FIG. 3b shows the optimum conditions of the proximity proteolysis reaction according to the present invention with respect to temperature and MgCl₂ concentration, FIG. 3c shows the optimal conditions of the nucleotide spacer between the target nucleic-acid binding sites of the ssDNA-protease conjugate and the ssDNA-zymogen conjugate according to the present invention, FIG. 3d shows the absorbance at 405 nm of various concentrations (0-40 nM) of target DNA-3, FIG. 3e shows the absorbance at 405 nm of various concentrations (0-40 nM) of target RNA, FIG. 3f shows effects of mouse serum (red) and HEK 293F lysate (yellow), as biological substrates, on proximal proteolysis reaction to detect RNA, and FIG. 3g shows effects of mouse serum (red) and HEK 293F lysate (yellow), as biological substrates, on proximal proteolysis reaction to detect RNA.

FIG. 4a a d FIG. 4b shows a proximity proteolysis reaction according to the present invention further including nucleic acid sequence-based amplification (NASBA), wherein FIG. 4a shows the use of NASBA to amplify a RNA transcript and to convert RNA through the proximity proteolysis reaction, and FIG. 4b shows the absorbance at 405 nm of various concentrations (0.01 pM to 10 pM) of the RNA transcript.

### DETAILED DESCRIPTION AND PREFERRED EMBODIMENTS OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

In the present invention, in an attempt to develop a method for detecting target nucleic acids that is fast, simple, and enables easy detection even at a small nucleic acid concentration, a proximity proteolysis reaction between a ssDNA-protease conjugate, in which ssDNA having a sequence complementary to the target nucleic acid is bound to a protease, and a ssDNA-zymogen conjugate, in which ssDNA having a sequence complementary to the target nucleic acid is bound to a zymogen, was used. As a result, it was found that nucleic acid detection is possible even at a concentration of about 100 pM, is realized through a one-step method of adding two DNA-protein conjugates and a colorimetric substrate to a sample, and is completed within 1 hour to detect the target nucleic acid.

Therefore, in one aspect, the present invention is directed to a method for detecting a target nucleic acid, the method including: (a) mixing a sample containing the target nucleic acid with a nucleic acid detection solution containing: i) a ssDNA-protease conjugate in which ssDNA having a sequence complementary to the target nucleic acid is bound to a protease; ii) a ssDNA-zymogen conjugate in which ssDNA having a sequence complementary to the target nucleic acid is bound to a zymogen; and iii) a substrate specific for the zymogen; and (b) detecting a signal generated by a proximity proteolysis reaction between the ssDNA-zymogen conjugate and the ssDNA-protease conjugate which are hybridized to the target nucleic acid.

As used herein, the term “protease” refers to an enzyme that hydrolyzes protein and peptide bonds.

As used herein, the term “zymogen”, which is interchangeable with “proenzyme”, refers to an inactive enzyme precursor, and is composed of an enzyme and an activity inhibitor protein of the enzyme bound through a peptide linker that can be cleaved by a protease. The zymogen is activated, when biochemical changes, such as revealing the active site of the enzyme, occur, by being cleaved into the enzyme and the activity inhibitor protein of the enzyme through hydrolysis or a change in configuration of the peptide linker through the protease.

As used herein, the term “target nucleic acid” refers to a nucleic-acid molecule to be detected by the method according to the present invention. The type of nucleic acid may be deoxyribonucleotide (DNA), ribonucleotide (RNA), and a mixture or combination thereof. The bases (nucleobases) constituting the same are nucleotides that are present in nature, such as guanine (G), adenine (A), thymine (T), cytosine (C), and uracil (U), but may include other natural and artificial modified bases. The term “modified base” refers to a base in which five nucleotides, namely guanine, adenine, thymine, cytosine and uracil, are chemically modified. In the present invention, the target nucleic acid needs to be a single strand when detected, but even a nucleic acid having a double strand or a higher-order structure may be used after conversion into a single strand by heat denaturation, alkali denaturation treatment or the like. The target nucleic acid of the present invention also includes aspects to which such denaturation treatment was added. In addition, the target nucleic acid of the present invention also includes cDNA produced through a reverse transcription reaction using RNA as a template.

As used herein, the term “sample” refers to a mixture predicted to contain a target nucleic acid to be detected. The sample is derived from living organisms including humans (e.g. blood, saliva, body fluids, body tissues, etc.), the environment [e.g., soil, sea water, environmental water (hot spring water, bathtub water, cooling tower water, etc.)], or artificial or natural materials (e.g. processed foods such as bread, fermented foods such as yogurt, cultivated plants such as rice or wheat, microorganisms, or viruses) and is usually subjected to a nucleic acid extraction operation. If necessary, the sample may be further subjected to nucleic acid purification.

As used herein, the term “oligonucleotide” refers to a linear oligomer formed by linking nucleotides including nucleotides such as adenosine, thymidine, cytidine, guanosine, and uridine, or modified nucleotides, through a phosphodiester bond, and represents DNA, RNA, and conjugates thereof. In some cases, the oligonucleotide may be a peptide nucleic acid (PNA).

As used herein, the term “complementary” refers to the state in which a polynucleotide or oligonucleotide strand is annealed with another strand to form a double-stranded structure and the nucleotide of each strand forms Watson-Click base pairing. Complementary nucleotides are generally A and T (or A and U), or C and G. The term “complementary” is also meant to include formation of non-Watson-click base pairing, such as pairing of modified nucleotides having deoxyinosine (dI) and 2-amino purine bases.

As used herein, the term “hybridization” generally refers to a reaction in which a single-stranded nucleic acid is bound to a complementary strand to form a double-stranded structure. DNA is usually a double strand, and when DNA is heated to a high temperature in a solution, the complementary hydrogen bond between the bases that form the double-strand is broken and DNA is cleaved into two single strands, which is called “denaturation”. The denatured single-stranded DNA is bound to a complementary base sequence again under appropriate conditions to form a double-strand, which is called “renaturation”. Hybrids may be formed between DNA-DNA, DNA-RNA or RNA-RNA. They may be formed between short strands and long strands that include regions complementary to the short strands. Incomplete hybrids may be formed, but as the incompleteness of hybrids increases, the possibility of formation of thereof decreases.

As used herein, the term “proximity proteolysis reaction” means that the distance between a protease and a peptide bond is close to one to five nucleotide spacers, so that the rate of proteolysis, in which the peptide bond of the protein is cleaved by hydrolysis to form an amino acid or a peptide, increases. The present inventors previously reported a β-lactamase zymogen, constructed by linking a permutate β-lactamase enzyme and a β-lactamase inhibitory protein (BLIP), which is an inhibitor protein thereof, through a linker that can be cleaved by a protease (H. Kim, et al., Chemical Communications 2014, 50, 10155-10157). In the present invention, a peptide linker including a TEV protease cleavage site was inserted between β-lactamase and BLIP. The TEV protease cleavage site is the cleavage site 1 of SEQ ID NO: 14 or the cleavage site 2 of SEQ ID NO: 15 (R. B. Kapust, et al., Protein Engineering vol. 14 no. 12, 993-1000, 2001). When the ssDNA-protease conjugate and the ssDNA-zymogen conjugate were hybridized to the target nucleic acid, β-lactamase could be activated by separating β-lactamase from BLIP through cleavage (degradation) of the peptide linker by the TEV protease. The activated β-lactamase hydrolyzes a substrate specific for β-lactamase to generate a signal.

TEV cleavage site 1: SEQ ID NO: 14: ENLYFQ/G

TEV cleavage site 2: SEQ ID NO: 15: ENLYFQ/S

(/: peptide bond cleaved by TEV protease)

When β-lactamase-specific substrate is CENTA (CENIA™ β-lactamase substrate), the change in absorbance at 405 nm, in the case where the ssDNA-protease conjugate and the ssDNA-zymogen conjugate are hybridized to the target nucleic acid, is increased compared to the change in absorbance at 405 nm, in the case where the conjugates are not hybridized thereto (Example 4).

When the β-lactamase-specific substrate is nitrocefin, in the case where the ssDNA-protease conjugate and the ssDNA-zymogen conjugate are not hybridized to the target nucleic acid, a yellow signal is shown, and in the case where the conjugates are hybridized to the target nucleic acid, a red signal is shown.

When the β-lactamase-specific substrate is CCF2-AM, upon emission of light having a wavelength of 408 nm thereto, in the case where the ssDNA-protease conjugate and ssDNA-zymogen conjugate are not hybridized to the target nucleic acid, light having a wavelength of 530 nm is emitted, and in the case where the conjugates are hybridized to the target nucleic acid, light having a wavelength of 460 nm is emitted.

When the β-lactamase-specific substrate is CCF4-AM, upon emission of light having a wavelength of 409 nm is emitted thereto, in the case where the ssDNA-protease conjugate and ssDNA-zymogen conjugate are not hybridized to the target nucleic acid, light having a wavelength of 520 nm is emitted, and in the case where the conjugates are hybridized to the target nucleic acid, light having a wavelength of 447 nm is emitted.

The present inventors have previously reported an engineered procaspase-3 that is activated by forming a dimer through proteolysis using a protease (D. K. Yang, et al., Anal. Methods, 2016, 8, 6270-6276). The enzyme activated by the protease hydrolyzed a substrate specific for caspase-3 to generate a signal.

In the present invention, the ssDNA (single-stranded DNA) is DNA having a single strand, and has a linear structure or a hairpin structure, but is not limited thereto. Linear ssDNA generated a signal more quickly than hairpin ssDNA in the detection of a target nucleic acid, and the reason for this is expected to be that linear ssDNA easily binds to the target nucleic acid.

In a specific embodiment of the present invention, the sequence of the ssDNA was selected from dual molecular beacons designed to target KRAS transcripts (P. J. Santangelo, et al., Nucleic Acid Research 2004, 32, e57).

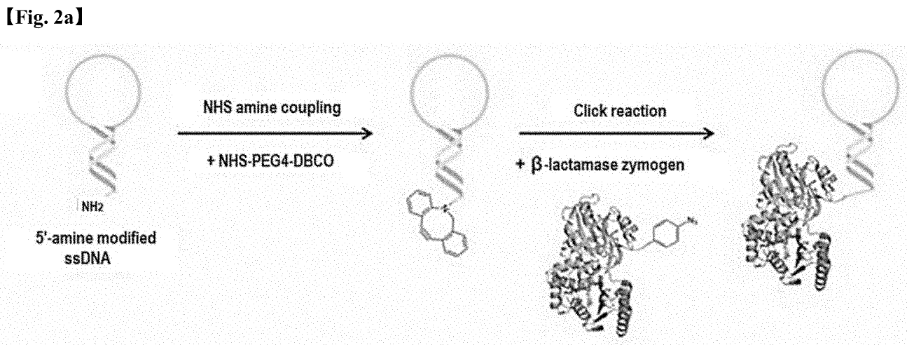

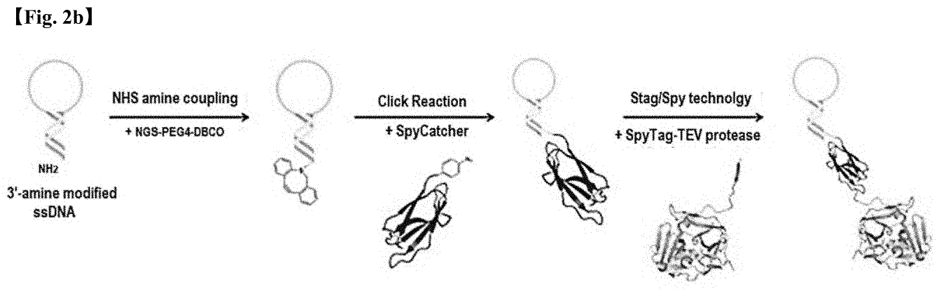

In the present invention, site-specific conjugation between β-lactamase zymogen and ssDNA was achieved through an accelerated click reaction between azide and cyclooctyne (in FIG. 2a)).

In the present invention, the TEV-ssDNA conjugate produced using a method similar to that used for β-lactamase zymogen exhibited remarkably lower activity compared to an unconjugated TEV protease. The loss of activity was expected to be caused by the purification procedure or covalent binding of ssDNA, and another strategy for binding TEV protease to ssDNA was used (FIG. 2b)). The SpyTag/Catcher system, which was first reported by Howarth et al., is based on an efficient isopeptide bond formation reaction between two proteins, SpyTag and SpyCatcher (M. Howarth, et al., Proceedings of the National Academy of Sciences of the United States of America 2012, 109, 690-697).

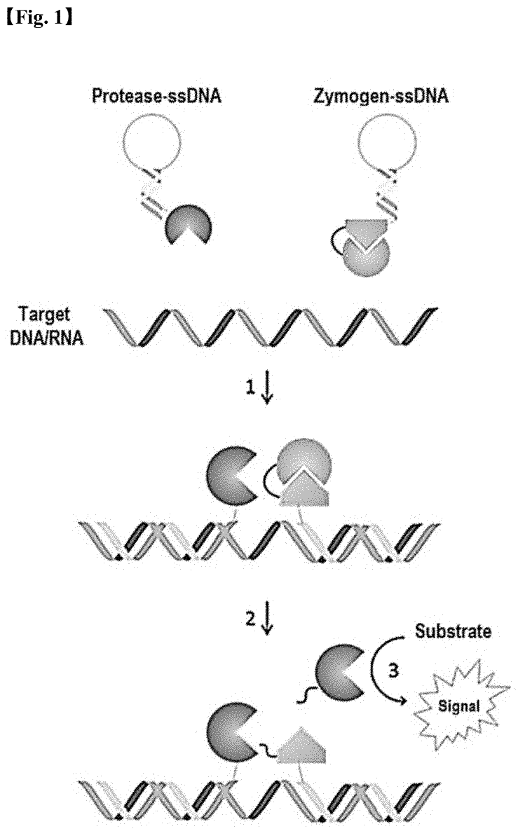

In the present invention, the proximity proteolysis reaction includes: (a) mixing a sample containing the target nucleic acid with a nucleic acid detection solution containing: i) a ssDNA-protease conjugate, in which ssDNA having a sequence complementary to the target nucleic acid is bound to a protease; ii) a ssDNA-zymogen conjugate, in which ssDNA having a sequence complementary to the target nucleic acid is bound to a zymogen; and iii) a substrate specific for the zymogen; (b) hybridizing the ssDNA-protease conjugate and the ssDNA-zymogen conjugate to the target nucleic acid; (c) hydrolyzing the zymogen using the protease; and (d) binding the enzyme activated by hydrolysis to a colorimetric substrate to generate a signal (FIG. 1), but is not limited thereto.

In the present invention, the proximity proteolysis reaction may be performed at a temperature of 20 to 40° C., preferably 25 to 35° C., and more preferably 37° C.

In a specific example of the present invention, the proximity proteolysis reaction was found to be the most optimal when the nucleic acid detection solution further contains 40 mM MgCl₂ and the proximity proteolysis reaction is performed at a temperature of 37° C. (FIG. 2b).

Although the method for detecting a target nucleic acid according to the present invention exhibits high sensitivity, some target nucleotides are present at a much lower concentration in a biological fluid. For example, viral RNA is present at a femtomolar concentration in the serum of a patient. In a specific embodiment of the present invention, in order to further improve the detection limit, isothermal RNA amplification and nucleic-acid-sequence-based amplification (NASBA) are applied to KRAS mRNA prepared by in-vitro transcription, but the invention is not limited thereto.

Therefore, in the present invention, step (a) may further include amplifying the target nucleic acid.

In another aspect, the present invention is directed to a nucleic acid detection solution containing: i) a ssDNA-protease conjugate, in which ssDNA having a sequence complementary to a target nucleic acid is bound to a protease; ii) a ssDNA-zymogen conjugate in which ssDNA having a sequence complementary to the target nucleic acid is bound to a zymogen; and iii) a colorimetric substrate specific for the zymogen.

In the present invention, the protease may be a tobacco etch virus (TEV) protease, a hepatitis C virus (HCV) protease, a tobacco vein mottling virus (TVMV) protease or a human rhinovirus (HRV) 3c protease, but is not limited thereto.

In the present invention, the zymogen may be β-lactamase zymogen or pro-caspase-3, but is not limited thereto.

In the present invention, the substrate may be a colorimetric or fluorescent substrate, but is not limited thereto.

In the present invention, the colorimetric substrate may be CENTA (CENTA™ β-lactamase substrate) or nitrocefin, which is a substrate specific for β-lactamase, but is not limited thereto.

In the present invention, the colorimetric substrate may be Ac-DEVD-pNA, Ac-DMQD-pNA, or Z-DEVD-pNA, which is a substrate specific for caspase-3, but is not limited thereto.

In the present invention, the fluorescent substrate may be CCF2-AM or CCF4-AM, which is a substrate specific for β-lactamase, but is not limited thereto.

In the present invention, the fluorescent substrate may be Ac-DEVD-AFC, Ac-DMQD-AMC or Z-DEVD-AFC, which is a substrate specific for caspase-3, but is not limited thereto.

In the present invention, the nucleic acid detection solution may further contain MgCl₂.

In the present invention, the concentration of MgCl₂ may be 10 mM to 90 mM, preferably 30 mM to 50 mM, and more preferably 40 mM.

Hereinafter, the present invention will be described in more detail with reference to examples. However, it will be obvious to those skilled in the art that these examples are provided only for illustration of the present invention and should not be construed as limiting the scope of the present invention.

### Example 1

### Plasmid Construction for Protein Expression

Tobacco etch virus (TEV) protease variants (L56V, S135G), which have been reported to exhibit improved solubility and stability compared to wild-type enzymes (LD Cabrita, et al., Protein science: a publication of the Protein Society 2007, 16, 2360-2367), were used. The synthetic gene of the TEV protease variants was cloned into pET-21a using EcoRI and XhoI, and then the double-stranded oligonucleotides of Strep-Tag and SpyTag were cloned into a plasmid containing the TEV protease gene using NdeI and EcoRI, and the cloned plasmid was called “pSPEL515”. The gene encoding the TEV protease variant is represented by SEQ ID NO: 1. A synthetic gene of SpyCatcher containing one TAG codon at the N-terminus of pSPEL515 was cloned into pET-21a using NdeI and XhoI to obtain pSPEL517. The gene encoding SpyCatcher is represented by SEQ ID NO: 2.

To construct a plasmid for expressing β-lactamase zymogen, pSPEL166, previously reported by the present inventors (H. Kim, et al., Chemical Communications 2014, 50, 10155-10157), was modified. The TAG codon was introduced by amplifying the β-lactamase zymogen gene using primer 1 of SEQ ID NO: 5 and primer 2 of SEQ ID NO: 6, and the PCR product was cloned into the same plasmid using NcoI and XhoI. The gene encoding β-lactamase zymogen is represented by SEQ ID NO: 3. Then, the cleavage site of the TEV protease was replaced with the original cleavage site of the MMP-2 protease using a double-stranded oligonucleotide regarding GGGSGGGSENLYFQ/GGGGSGGGS (/: peptide bond cleaved by TEV protease) through BamHI and HindIII.

Primer 1: SEQ ID NO: 5:
AACCTTCCATGGGCTAGGGCGGCAGCGGTGGTAGCGCGGGGGTGATGACC
GGGGCG
Primer 2: SEQ ID NO: 6:
AACCTTCTCGAGTGCCTGACTCCCCGTCGTGTAGATAACTACGATACG

### Example 2

Protein Expression and Purification

1. SpyTag-TEV Protease

E. coli BL21 (DE3) cells transformed with pSPEL515 were used for SpyTag-TEV protease expression. Recombinant E. coli strains were cultured at 2×YT at 37° C. until an optical density, that is, an OD₆₀₀, reached 0.5. Protein expression was induced with 0.4 mM β-D-1-thiogalactopyranoside (IPTG) at 25° C. for 8 hours. Cell pellets were obtained by centrifugation and then stored at −20° C. until purification. SpyTag-TEV protease having a His6-tag at the N-terminus thereof was purified using a Ni-NTA resin (Clontech, USA) according to the manufacturer's instructions. Purified SpyTag-TEV protease was stored in a TEV protease storage buffer (50 mM Tris, 10 mM NaCl, 0.5 mM EDTA, 40% (v/v) glycerol, pH 8.0) at −20° C.

2. SpyCatcher

In order to introduce 4-azido-L-phenylalanine (AzF) at the amber codon position of the SpyCatcher protein, pSEPL517 was transformed into E. coli BL21 (DE3) cells having two different plasmids: pSPEL150 expressing an orthogonal pair of tRNA and aminoacyl-tRNA synthetase of Methanococcus jannaschii to introduce AzF in response to a TAG codon (AzF-RS/tRNACUA), and pSPEL168 overexpressing E. coli prolyl-tRNA synthetase (ProRS) to prevent AzF from being mistaken for the Pro position of the protein. The cells were cultured in 2×YT at 37° C. until OD₆₀₀ reached 0.5, and then 0.2% L-arabinose and 50 nM anhydrous tetracycline (aTc) were each added to induce the expression of orthogonal aminoacyl-tRNA synthetase and ProRS. When the OD₆₀₀ reached 1.0, 0.4 mM IPTG was added in the presence of 1 mM AzF to induce the expression of SpyCatcher at 30° C. for 8 hours. The SpyCatcher purification process is the same as the SpyTag-TEV purification process. The purified protein was stored in a storage buffer (70 mM NaCl, 1.5 mM KCl, 5 mM Na₂HPO₄, 1 mM KH₂PO₄, 20% (V/V) glycerol, pH 7.4) at −20° C.

3. β-lactamase Zymogen

E. coli BL21 (DE3) transformed with three plasmids (pSPEL427, pSEPL150 and pSPEL168) was used to express β-lactamase zymogen having AzF. The expression of AzF-RS and ProRS was induced at an OD₆₀₀ of 0.5 using 0.26% L-arabinose and 50 nM aTc, respectively, and then expression of β-lactamase zymogen was induced using 0.4 mM IPTG in the presence of 1 mM AzF at 25° C. for 16 hours. The protein was purified from the periplasmic fraction according to the method described above. The purified β-lactamase zymogen was stored in a storage buffer at −20° C.

4. Determination of Purified Protein Concentration

The purified protein concentration was determined by measuring the absorbance at 280 nm using the extinction coefficient calculated at the ProtParam site (http://web.expasy.org/protparam/).

### Example 3

Conjugation of Single-Stranded DNA (ssDNA) and Protein

1. Derivatization of ssDNA by N-hydroxysuccinimide ester-(polyethyleneglycol)4-dibenzylcyclooctyne (NHS-PEG4-DECO)

ssDNA functionalized with a 5′-amine group (ssDNA-1 or ssDNA-2) or a 3′-amine group (ssDNA-3) was purchased from Bioneer Co. (Korea). ssDNA was mixed with a 20-fold molar excess of NHS-PEG4-DECO linker, and the reaction was conducted at 25° C. in a phosphate-buffered saline solution (PBS; 137 mM NaCl, 2.7 mM KCl, 10 mM Na₂HPO₄, 2 mM KH₂PO₄, pH 7.4) in the dark for 2 hours. The modified ssDNA was precipitated with ethanol to remove an excess linker, and the pellet was resuspended in PBS for storage at −20° C.

ssDNA-1: SEQ ID NO: 11:
[Amine]CCTACGCCACCAGCTCCGTAGG
ssDNA-2: SEQ ID NO: 12:
[Amine]CCTACGCCACCAGC
ssDNA-3: SEQ ID NO: 13:
AGTGCGCTGTATCGTCAAGGCACT[Amine]

2. ssDNA-β-lactamase Zymogen Conjugate

The ssDNA (ssDNA-1 or ssDNA-2) having a modified 5′-end was mixed with a β-lactamase zymogen protein containing AzF at a molar ratio of 5:1 in PBS, and the mixture was incubated at 4° C. for 16 minutes. First, the unconjugated protein was removed through anion-exchange chromatography using a HiTrap Q column (GE Healthcare Life Sciences, USA). A mixture of ssDNA-conjugate and ssDNA was eluted with a 0.2-1M NaCl gradient. The eluted fraction was further purified by gel filtration chromatography using a Superdex-column (GE Healthcare Life Sciences, USA) to remove ssDNA. The purified ssDNA-β-lactamase zymogen conjugate was stored in a storage buffer at −20° C.

3. ssDNA-TEV Protease Conjugate

First, the SpyCatcher protein containing AzF was conjugated to ssDNA (ssDNA-3) having a modified 3′ end using an NHS-PEG4-DBCO linker. The protein was mixed with a 5-fold molar excess of modified ssDNA in PBS, and the mixture was incubated at 25° C. for 4 hours. Unconjugated SpyCatcher was removed through anion-exchange chromatography using a HiTrap Q column. The partially purified ssDNA-SpyCatcher conjugate containing unreacted ssDNA was reacted with a SpyTag-TEV protease in PBS at 4° C. for 2 hours. Because the modified ssDNA was not expected to interfere with the reaction between SpyTag and SpyCatcher, the conjugation reaction was performed in the presence of unreacted ssDNA. The ssDNA-TEV protease conjugate was purified using Strep-Tactin resin (IBA Lifesciences, Germany) according to the manufacturer's instructions. Purified ssDNA-TEV protease in a TEV protease storage buffer was stored at −20° C.

4. Determination of Protein and DNA Concentration in ssDNA-Protein Conjugate

The concentration of the conjugate was calculated by measuring the absorbance at 260 and 280 nm using the following equation. The DNA extinction coefficient (ε_{260,DNA}) at 260 nm was calculated using molbiotools (http://www.molbiotools.com/dnacalculator.html), and the extinction coefficient at 280 nm (ε_{280,DNA}) was determined by measuring the absorbance of a sample having a known concentration. The protein extinction coefficient ε_{280,protein}) at 280 nm was calculated at the ProtParam site, and the protein extinction coefficient (ε_{260,protein}) at 260 nm was determined by measuring the absorbance of a sample having a known concentration.

A₂₆₀=A_{260,DNA}++A_{260,protein}=ε_{260 DNA}×b×C_{DNA}+ε_{260, protein}×b×Cₚᵣₒₜₑᵢₙ

A₂₆₀=A_{260,DNA}++A_{280,protein}=ε_{280 DNA}×b×C_{DNA}+ε_{280,protein}×b×Cₚᵣₒₜₑᵢₙ

ε: Extinction coefficient (M⁻¹cm⁻¹)

b: Path leng (cm)

C: Concentration (M)

### Example 4

Detection of Nucleic Acid by Proximity Proteolysis Reaction

1. Experimental Method

A proximity proteolysis reaction was initiated by adding 40 nM ssDNA-TEV protease, 20 nM ssDNA-β-lactamase zymogen and 200 μM CENTA (CENTA™ β-lactamase substrate, EMD Millipore, Billerica, Mass., USA) to a sample solution containing the target nucleotide molecule in the presence of a reaction buffer (137 mM NaCl, 2.7 mM KCl, 10 mM Na₂HPO₄, 2 mM KH₂PO₄, 40 mM MgCl₂, 10 mM DTT, 0.5% (w/v) BSA, pH 7.4). The target nucleotide was established using a 46-nt DNA oligonucleotide (target DNA-4) sequence corresponding to a portion of the KRAS transcript. The reaction was performed at 37° C., and the incubation time was 45 minutes for ssDNA detection and 60 minutes for RNA detection. For RNA samples, a 10 U/l mL RNase inhibitor (Roche, Switzerland) was added. Hydrolysis of CENTA by β-lactamase was observed through absorbance at 405 nm, measured using a plate reader (Synergy HT Multi-Detection Reader; BioTek Instruments, USA). The limit of detection (LOD) was calculated using a standard curve as the target concentration of the absorbance, corresponding to the sum of the target mean absorbance and three times the standard deviation. The interference of biological substrates in proximity proteolysis assay was tested using two biological fluids: HEK 293F cell lysate and mouse serum (Sigma, USA). A HEK 293F cell lysate was prepared through ultrasonication using a 130-watt ultrasonic disperser (Sonics & Materials, Inc., USA), and a 200 μL analytic sample was prepared using 1×10⁶ of the cell lysate. Mouse serum was added to the sample at a concentration of 5% (v/v).

2. Analysis of Optimal Conditions for Proximity Proteolysis Reaction

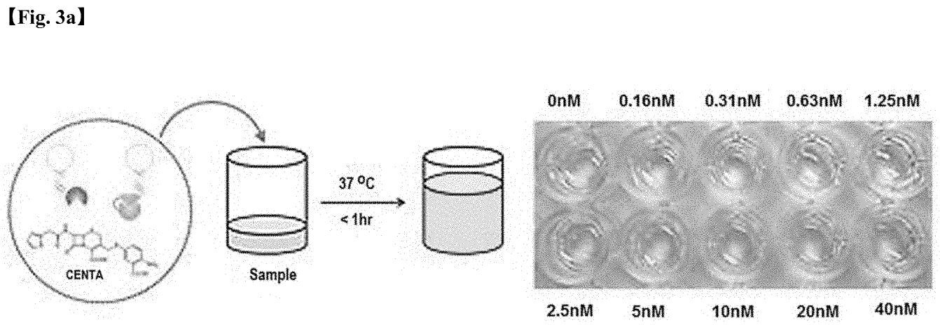

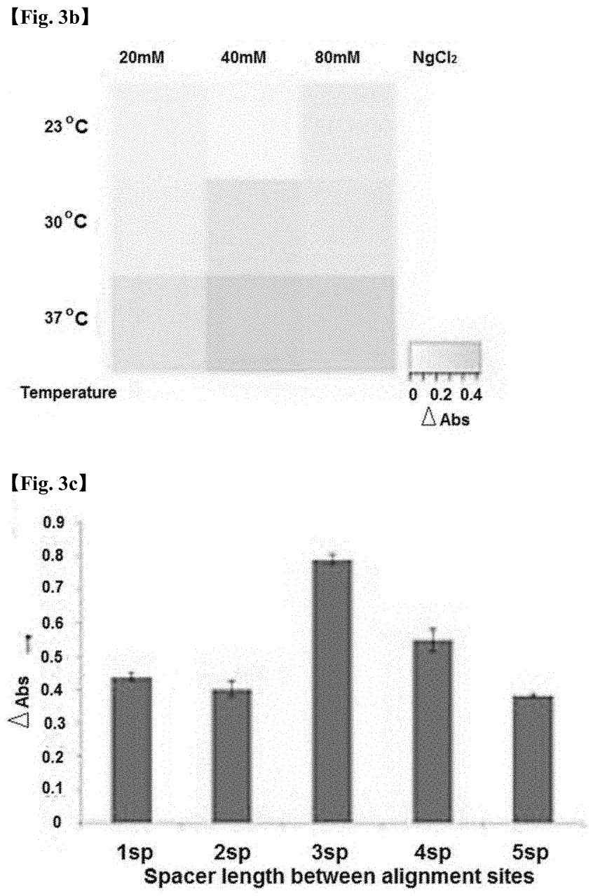

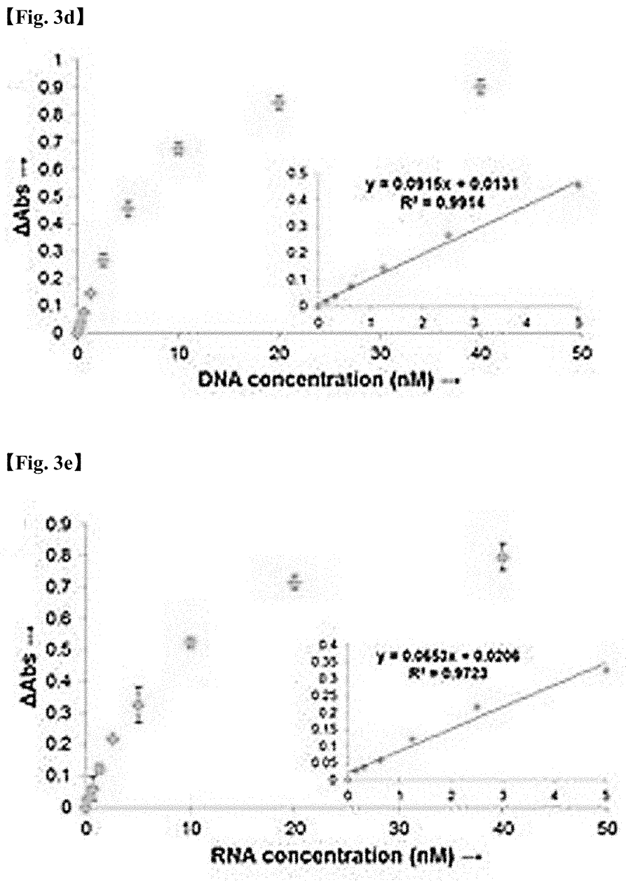

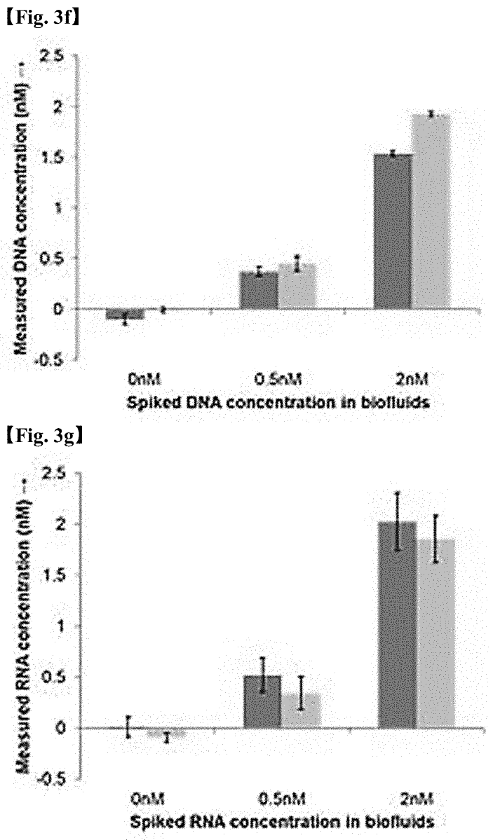

In order to establish optimal conditions for the proximity proteolysis reaction, the signal difference depending on the concentration of MgCl₂ and temperature conditions was analyzed. Initially, a relatively small signal difference was observed depending on the presence of the target nucleotide (20 mM MgCl₂ at 25° C.). Two factors of the MgCl₂ concentration and temperature were further optimized, and the conditions of 40 mM MgCl₂ and 37° C. showed the highest signal difference (FIG. 3b).

Differences in proteolytic reactions depending on the spatial arrangement of target nucleic-acid binding sites of TEV protease and β-lactamase zymogen were analyzed. A proximity proteolysis reaction was performed with 1 to 5 nucleotide spacers (target DNA 1 to 5) as the distance between the binding sites of the template DNA regarding two ssDNAs. As a result, the three-nucleotide spacer showed a higher signal difference than other cases (FIG. 3c).

Target DNA-1:
TACGGAGCTGGTGGCGTAGGtAGTGCCTTGACGATACAGCGCA
Target DNA-2:
TAGGGAGCTGGTGGCGTAGGtaAGTGCCTTGACGATACAGCGCA
Target DNA-3:
TACGGAGCTGGTGGCGTAGGtagAGTGCCTTGACGATACAGCGCA
Target DNA-4:
TACGGAGCTGGTGGCGTAGGtagaAGTGCCTTGACGATACAGCGCA
Target DNA-5:
TACGGAGCTGGTGGCGTAGGtagatAGTGCCTTGACGATACAGCGCA
Target RNA:
LACGGAGCUGGUGGCGUAGGuagAGUGCCUUGACGAUAEAGCGCA

(The underlined sequence represents the nucleotide spacer between the two binding sites of β-lactamase zymogen-ssDNA and TEV-ssDNA.)

3. Experiment Result

Proximal proteolysis reactions were conducted at various concentrations of target DNA oligonucleotides under optimized conditions. As shown in FIG. 3d), a difference in the amount of color development to yellow due to a change in absorbance at 405 nm was observed depending on the DNA concentration immediately after the protein-ssDNA conjugate and CENTA were added. The change in absorbance at 405 nm was 0.166 in the absence of the target nucleic acid, and the change in absorbance at 405 nm was 1.019 in the presence of the target nucleic acid. The highest signal difference was observed at 45 minutes. In this case, absorbance at 405 nm was shown compared to the target concentration. A hyperbolic curve appeared at all concentrations in the experimental range, and a linear relationship was observed from 94 pM as the limit of detection (LOD) to 5 nM (FIG. 3d).

| Target (nM) | 40 | 20 | 10 | 5 | 2.5 | 1.25 | 0.625 | 0.313 | 0.156 | 0 |
|---|---|---|---|---|---|---|---|---|---|---|
| Absorbance | 1.341 | 1.282 | 1.115 | 0.894 | 0.705 | 0.583 | 0.515 | 0.477 | 0.455 | 0.440 |
| at 405 nm | | | | | | | | | | |

Since ssDNA bound to TEV protease and β-lactamase zymogen was originally produced to detect KRAS mRNA, proximity proteolysis analysis was applied to the synthesized RNA nucleotides corresponding to the DNA targets used above. Because the interaction between DNA and RNA is weak, it took to develop color of ssDNA longer than the DNA target (45 minutes). A hyperbolic curve was observed over the entire range of target concentration, and a linear relationship was observed from 93 pM as a limit of detection to 5 nM (FIG. 3e).

The interference of biological substrates in proximity proteolysis was evaluated using HEK293F cell lysate and mouse serum, and the results showed that the proximity proteolysis can be used to detect nucleotides of DNA and RNA present in the biological sample, as shown in FIGS. 3f and 3g.

In particular, it was found that the proximity protein hydrolysis method was not only simple to use, but also took less than 1 hour to detect the target nucleotide at concentrations smaller than a nanomolar concentration.

### Example 5

Nucleic-Acid-Sequence-Based Amplification (NASBA)

The synthetic gene of KRAS was cloned into pET-21a (IDT, USA) using NdeI and XhoI (pSPEL570), and a PCR fragment for transcription was prepared using primer 3 of SEQ ID NO: 7 and primer 4 of SEQ ID NO: 8. The gene encoding KRAS is represented by SEQ ID NO: 4. The KRAS transcript was produced by in-vitro transcription using the EZ High-Yield In-Vitro Transcription Kit (Enzynomics, Korea) according to the manufacturer's instructions. RNA was purified using a MEGAclear Kit (Ambion, USA) and stored at −20° C. KRAS mRNA was amplified through NASBA reaction using primer 5 of SEQ ID NO: 9 and primer 6 of SEQ ID NO: 10, and a NASBA Liquid Kit Complete (Life Sciences Advanced Technologies, USA) according to the manufacturer's instructions, and RNA fragments were used for proximity proteolysis reaction.

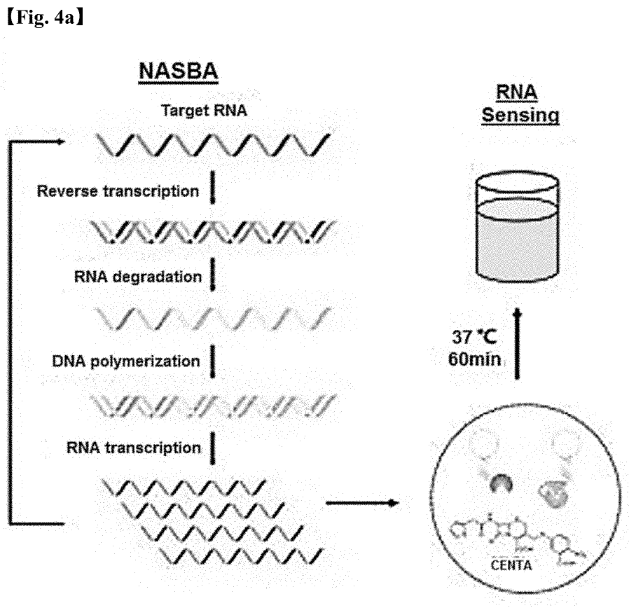

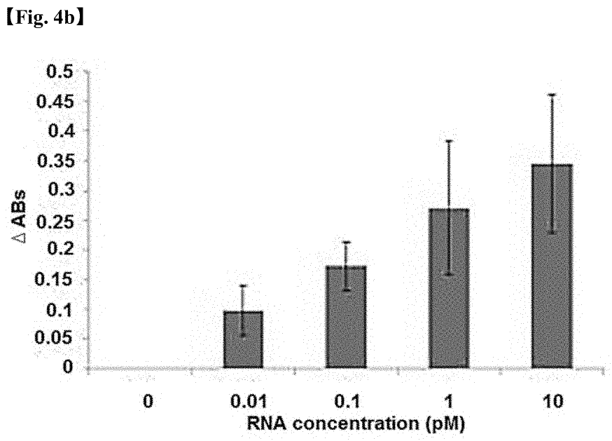

A completely different signal from the baseline was observed in the sample containing the KRAS transcript concentration as low as 10 fM, and was found to be 10,000 times lower than the detection limit without amplification (FIG. 4b).

Primer 3: SEQ ID NO: 7:
TCGATCCCGCGAAATTAATACGACTCACTATAGG
Primer 4: SEQ ID NO: 8:
CAAAAAACCCCTCAAGACCCGTTTA
Primer 5: SEQ ID NO: 9:
AATTCTAATACGACTCACTATAGGGAGAAGGCTCGCTTGCGCGAATACGG
AGCTGGTGGCG
Primer 6: SEQ ID NO: 10:
GTCGTATCCAGTGCGTCATCTTTCGAGGTGACTTGCACTGGATACGACTG
CGCT

### INDUSTRIAL APPLICABILITY

The method for detecting a target nucleic acid according to the present invention is carried out through a one-step process of adding two DNA-protein conjugates and a colorimetric substrate to a sample using a proximity proteolysis reaction, and takes less than an hour to detect a target nucleic acid, and thus is quick, simple and highly sensitive. Thus, the method will be useful in disease diagnosis, testing of genetically modified organisms (GMOs), and forensic medicine investigations that require detection of target nucleic acids.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

[Sequence Listing Free Text]

An electronic file is attached.

1. A method for detecting a target nucleic acid, the method comprising:
(a) mixing a sample containing the target nucleic acid with a nucleic acid detection solution containing:
i) a ssDNA-protease conjugate in which ssDNA having a sequence complementary to the target nucleic acid is bound to a protease;
ii) a ssDNA-zymogen conjugate in which ssDNA having a sequence complementary to the target nucleic acid is bound to a zymogen; and
iii) a substrate specific for the zymogen; and
(b) detecting a signal generated by a proximity proteolysis reaction between the ssDNA-zymogen conjugate and the ssDNA-protease conjugate which are hybridized to the target nucleic acid.

2. The method according to claim 1, wherein the zymogen comprises an enzyme and an activity inhibitor protein of the enzyme bound through a peptide linker that can be cleaved by a protease.

3. The method according to claim 1, wherein the proximity proteolysis reaction in step (b) comprises:
cleaving the zymogen into the enzyme and the activity inhibitor protein of the enzyme by cleaving the peptide linker by the protease, when the ssDNA-protease conjugate and the ssDNA-zymogen conjugate are hybridized to the target nucleic acid, to activate the enzyme; and
hydrolyzing the substrate by the activated enzyme to generate a signal.

4. The method according to claim 1, wherein the protease is a tobacco etch virus (TEV) protease, a hepatitis C virus (HCV) protease, a tobacco vein mottling virus (TVMV) protease or a human rhinovirus (HRV) 3c protease.

5. The method according to claim 1, wherein the zymogen is β-lactamase zymogen or pro-caspase-3.

6. The method according to claim 1, wherein the substrate is a colorimetric or fluorescent substrate.

7. The method according to claim 6, wherein when the zymogen is β-lactamase zymogen and the colorimetric substrate is CENTA, a change in absorbance at 405 nm, in case where the ssDNA-protease conjugate and the ssDNA-zymogen conjugate are hybridized to the target nucleic acid, is increased compared to a change in absorbance at 405 nm, in case where the conjugates are not hybridized thereto.

8. The method according to claim 6, wherein, when the zymogen is β-lactamase zymogen and the colorimetric substrate is nitrocefin, in case where the ssDNA-protease conjugate and the ssDNA-zymogen conjugate are not hybridized to the target nucleic acid, a yellow signal is shown, and in case where the conjugates are hybridized to the target nucleic acid, a red signal is shown.

9. The method according to claim 6, wherein, when the zymogen is β-lactamase zymogen and the fluorescent substrate is CCF2-AM, upon emission of light having a wavelength of 408 nm thereto, in case where the ssDNA-protease conjugate and ssDNA-zymogen conjugate are not hybridized to the target nucleic acid, light having a wavelength of 530 nm is emitted, and in case where the conjugates are hybridized to the target nucleic acid, light having a wavelength of 460 nm is emitted.

10. The method according to claim 6, wherein when the zymogen is β-lactamase zymogen and the fluorescent substrate is CCF4-AM, upon emission of light having a wavelength of 409 nm thereto, in case where the ssDNA-protease conjugate and ssDNA-zymogen conjugate are not hybridized to the target nucleic acid, light having a wavelength of 520 nm is emitted, and in case where the conjugates are hybridized to the target nucleic acid, light having a wavelength of 447 nm is emitted.

11. The method according to claim 1, wherein the nucleic acid detection solution in step (a) further comprises MgCl₂.

12. The method according to claim 1, wherein the proximity proteolysis reaction is performed at a temperature of 20 to 40° C.

13. The method according to claim 1, wherein step (a) further comprises amplifying the target nucleic acid.

14. The method according to claim 11, wherein a concentration of MgCl₂ is 10 mM to 90 mM.

15. A nucleic acid detection solution comprising:
i) a ssDNA-protease conjugate, in which ssDNA having a sequence complementary to a target nucleic acid is bound to a protease;
ii) a ssDNA-zymogen conjugate in which ssDNA having a sequence complementary to the target nucleic acid is bound to a zymogen; and
iii) a substrate specific for the zymogen.

16. The nucleic acid detection solution according to claim 15, wherein the protease is a tobacco etch virus (TEV) protease, a hepatitis C virus (HCV) protease, a tobacco vein mottling virus (TVMV) protease or a human rhinovirus (HRV) 3c protease.

17. The nucleic acid detection solution according to claim 15, wherein the zymogen comprises an enzyme and an activity inhibitor protein of the enzyme bound through a peptide linker that can be cleaved by a protease.

18. The nucleic acid detection solution according to claim 15, wherein the zymogen is β-lactamase zymogen or pro-caspase-3.

19. The nucleic acid detection solution according to claim 15, wherein the substrate is a colorimetric or fluorescent substrate.

20. The nucleic acid detection solution according to claim 15, wherein the nucleic acid detection solution further comprises MgCl₂.

21. The nucleic acid detection solution according to claim 20, wherein a concentration of MgCl₂ is 10 mM to 90 mM.


SEQUENCE LISTING (15 sequences)

SEQ ID NO: 1 - DNA, 867 bp, Artificial Sequence
  SpyTag-TEV
atgggcagcagccatcatcatcatcatcacggatcctggagccacccgcagttcgaaaaaaagcttggtggcggttctggtggtggcagcgcccacatcgtgatggtggacgcctacaagccgacgaagggtggcggcagcggcggcggtagcgaattcgaaagcctgtttaagggcccgcgtgattacaacccgatctcttctaccatctgccatctgaccaacgagtccgacggtcacaccacctctctgtatggcatcggttttggtccattcatcatcacgaacaaacacctgttccgtcgtaacaacggtaccctcgtggtgcagagcctgcacggtgtatttaaggttaagaacacgacgacgctccaacagcatctcattgatggccgcgacatgattatcatccgtatgccgaaggatttcccgccgttcccgcagaaactgaaattccgtgaaccgcagcgtgaagaacgtatctgcctcgttaccacgaactttcagaccaagagcatgtcctctatggtttccgacacgtcctgtaccttcccgagcggcgacggcattttctggaagcactggattcagacgaaagacggccagtgtggttctccgctggtaagcacccgtgacggcttcattgtcggtatccactccgcgtccaatttcaccaacaccaacaactacttcacgtccgtaccgaagaacttcatggaactcctgacgaaccaggaagcgcagcagtgggtatctggttggcgtctcaacgcggattctgttctgtggggtggtcacaaggtgttcatggttaaaccggaagaaccgttccagccagttaaggaagcgactcaactgatgaattga

SEQ ID NO: 2 - DNA, 429 bp, Artificial Sequence
  SpyCather
atgggcagcagccatcatcatcatcatcacggatccggcggcggttcttagaagcttggtggcggttctggtggtggcagcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatttaa

SEQ ID NO: 3 - DNA, 1431 bp, Artificial Sequence
  beta-lactamase zymogen
atgggctagggcggcagcggtggtagcgcgggggtgatgaccggggcgaagttcacgcagatccagttcgggatgacacgtcagcaggtcctcgacatagccggtgcggagaactgtgagaccggcgggtcgttcggggacagcatccactgccgggggcacgcggcaggggactactacgcctacgccaccttcggcttcaccagcgccgccgccgacgcgaaggtggactcgaagagccaggagaagctgctggccccgagcgccccgacgctcaccctcgccaagttcaaccaggtcaccgtggggatgaccagggcccaggtactggcgaccgtcgggcaggggtcctgcaccacctggagtgagtactacccggcctatccgtcgacggccggggtgaccctcagcctgtcctgcttcgatgtggacggttactcgtcgacgggggcctaccgaggctcggcgcacctctggttcacggacggggtgcttcagggcaagcggcagtgggaccttgtaggatccggcggcggtagcggtggcggcagcgaaaacctgtattttcagggcggcggcggcagcggcggtggtagcaagcttatggacgagcgtaaccgtcaaattgcggaaatcggcgcatctctgatcaaacactggggtggcggcggtggccacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtgagccctcccgtatcgtagttatctacacgacggggagtcaggcactcgagcaccaccaccaccaccactga

SEQ ID NO: 4 - DNA, 596 bp, Artificial Sequence
  KRAS
atgactgaatataaacttgtggtatacggagctggtggcgtaggtagagtgccttgacgatacagcgcattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattctcgagaacaaattaaaagagttaaggactctgaagatgtacctatggtcctagtagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtagcggccgcactcgagcaccaccaccaccaccactga

SEQ ID NO: 5 - DNA, 56 bp, Artificial Sequence
  Primer 1
aaccttccatgggctagggcggcagcggtggtagcgcgggggtgatgaccggggcg

SEQ ID NO: 6 - DNA, 48 bp, Artificial Sequence
  Primer 2
aaccttctcgagtgcctgactccccgtcgtgtagataactacgatacg

SEQ ID NO: 7 - DNA, 34 bp, Artificial Sequence
  Primer 3
tcgatcccgcgaaattaatacgactcactatagg

SEQ ID NO: 8 - DNA, 25 bp, Artificial Sequence
  Primer 4
caaaaaacccctcaagacccgttta

SEQ ID NO: 9 - DNA, 61 bp, Artificial Sequence
  Primer 5
aattctaatacgactcactatagggagaaggctcgcttgcgcgaatacggagctggtggcg

SEQ ID NO: 10 - DNA, 54 bp, Artificial Sequence
  Primer 6
gtcgtatccagtgcgtcatctttcgaggtgacttgcactggatacgactgcgct

SEQ ID NO: 11 - DNA, 22 bp, Artificial Sequence
  ssDNA-1
cctacgccaccagctccgtagg

SEQ ID NO: 12 - DNA, 14 bp, Artificial Sequence
  ssDNA-2
cctacgccaccagc

SEQ ID NO: 13 - DNA, 24 bp, Artificial Sequence
  ssDNA-3
agtgcgctgtatcgtcaaggcact

SEQ ID NO: 14 - protein, 7 aa, Artificial Sequence
  TEV cleavage site 1
ENLYFQG

SEQ ID NO: 15 - protein, 7 aa, Artificial Sequence
  TEV cleavage site 2
ENLYFQS